(19) United States
(12) Patent Application Publication
(10) Pub. No.: US 2026/0124320 A1
(43) Pub. Date: May 7, 2026

(54) FORMULATIONS FOR AAV GENE THERAPY

(71) Applicant: MeiraGTx UK II Limited, London (GB)

(72) Inventors: Jayan Senaratne, London (GB); Greta Jasulaityte, London (GB)

(21) Appl. No.: 19/117,759

(22) PCT Filed: Oct. 11, 2023
(86) PCT No.: PCT/IB2023/060240

§ 371 Date: Apr. 2, 2025

Related U.S. Application Data
(60) Provisional application No. 63/379,117, filed on Oct. 11, 2022.

(51) Int. Cl. A61K 48/00; A61K 47/02; A61K 47/26; A61K 47/40; C12N 15/86

(52) U.S. Cl. CPC A61K 48/0033; A61K 47/02; A61K 47/26; A61K 47/40; A61K 48/0091; C12N 15/86; C12N 2750/14143

(57) ABSTRACT

Compositions for the storing and maintaining virus viability including a balance of ionic strength and osmolality, which lead to reproducible and stable product quality at room temperature, at 4° C., and have the ability to withstand 10 freeze/thaw events.

### CROSS-REFERENCE TO RELATED APPLICATIONS

This application claims the benefit under 35 U.S.C. § 119(e) of the earlier filing date of U.S. Provisional Patent Application No. 63/379,117, filed Oct. 11, 2022, which is hereby incorporated by reference in its entirety.

### FIELD

The present disclosure relates to formulations for storing and maintaining the viability or infectivity of adeno-associated virus (AAV) following freeze-thawing cycles.

### BACKGROUND

AAV gene therapies in a liquid formulation are rarely considered stable under refrigeration and are typically stabilized through freezing at −80° C. However, the process of freezing and thawing may compromise product quality, while transportation and storage of frozen materials is often complicated and expensive. Furthermore, the use of freezing at −80° C. reduces the number of available drug delivery device options, e.g., pre-filled syringes, which can hinder accessibility of the product. As such, new formulations for storing and maintaining viability/infectivity of AAV gene therapy products are urgently needed.

### SUMMARY

Embodiments of this disclosure are directed to formulations that maintain AAV product quality at various storage temperatures, from about ambient temperature including up to −80° C., and/or exposure to multiple freeze/thaw conditions over time.

Accordingly, in certain aspects, provided is a formulation for an AAV particle, wherein the formulation comprises a saccharide, a buffer, a surfactant, a salt or combinations thereof. In certain embodiments, the saccharide comprises one or more saccharides. In certain embodiments, the one or more saccharide comprise trehalose, cyclodextrin, sucrose or combinations thereof. In certain embodiments, the salt comprises a sodium salt, a magnesium salt, a calcium salt, a potassium salt, a phosphate salt, a sulfate salt, triethylamine, guanidine, N-substituted guanidine salts, acetamidine, N-substituted acetamidine, pyridine, picoline, ethanolamine, triethanolamine, dicyclohexylamine or N,N′-dibenzylethylenediamine salts, or combinations thereof. In certain embodiments, the sodium salt comprises sodium chloride, sodium phosphate, or the combination thereof. In certain embodiments, the magnesium salt is magnesium sulfate. In certain embodiments, the salt comprises sodium chloride, sodium phosphate, magnesium sulfate or the combination thereof. In certain embodiments, the formulation further comprises a buffer, comprising phosphate buffered saline (PBS) sodium phosphate, citric acid, acetic acid, tromethamine, aspartic acid, glutamic acid, HEPES, Tris, Bicine, acetate, glutamate, lactate, maleate, tartrate, phosphate, citrate, carbonate, glycinate, histidine, glycine, lysine, arginine, succinate, HEPES (4-(2-hydroxyethyl)-1-piperazineethanesulfonic acid), MOPS (3-(N-morpholino) propanesulfonic acid), MES (2-(N-morpholino)ethanesulfonic acid), triethanolamine buffer, and combinations thereof. In certain embodiments, the surfactant is a non-ionic surfactant. In certain embodiments, the non-ionic surfactant comprises a polysorbate. In certain embodiments, the polysorbate is polysorbate 80. In certain embodiments, the formulation comprises an ionic strength from about 100 mM to about 700 mM. In certain embodiments, the formulation comprises an osmolality (mOsm/kg) from about 100 mOsm/kg to about 800 mOsm/kg. In certain embodiments, the formulation comprises a pH of about 7.0 to about 8.0. In certain embodiments, the formulation comprises a pH of about 7.5. In certain embodiments, the formulation is utilized for storing or maintaining viability of adeno-associated virus (AAV) particles over periods of time at temperatures from about 20° C. to about −80° C. In certain embodiments, the AAV particles are stable over multiple freeze-thawing cycles. In certain embodiments, the AAV particles are derived from an AAV with an AAV serotype selected from serotypes 1, 2, 3, 4, 5, 6, 7, 8, 9, 10, 11, 12, 13, 14, 15, 16, DJ or DJ/8. In certain embodiments, the AAV particle comprises a genome derived from AAV serotype 2. In certain embodiments, the AAV particles comprise a capsid derived from AAV serotype 2.

In another aspect, a pharmaceutical composition for storing, maintaining infectivity, and/or maintaining viability of AAV particles comprises a cryoprotectant, a buffer, a surfactant and a salt. In certain embodiments, the cryoprotectant comprises one or more saccharides. In certain embodiments, the saccharides comprise trehalose, cyclodextrin, sucrose or combinations thereof. In certain embodiments, the salt comprises a sodium salt, a magnesium salt, a calcium salt, a potassium salt, a phosphate salt, a sulfate salt or combinations thereof. In certain embodiments, the sodium salt comprises sodium chloride, sodium phosphate or the combination thereof. In certain embodiments, the magnesium salt is magnesium sulfate. In certain embodiments, the surfactant is a non-ionic surfactant. In certain embodiments, the non-ionic surfactant comprises a polysorbate. In certain embodiments, the polysorbate is polysorbate 80. In certain embodiments, the pharmaceutical composition comprises an ionic strength from about 100 mM to about 700 mM. In certain embodiments, the pharmaceutical composition comprises an osmolality (mOsm/kg) from about 100 mOsm/kg to about 800 mOsm/kg. In certain embodiments, the pharmaceutical composition comprises a pH of about 7.0 to about 8.0. In certain embodiments, the pharmaceutical composition comprises a pH of about 7.5. In certain embodiments, the pharmaceutical composition is utilized for storing or maintaining viability of an AVV particle over periods of time at temperatures from about 20° C. to about −80° C. In certain embodiments, the AAV particle remains stable over multiple freeze/thaw cycles.

In another aspect, a cryoprotective formulation for storing, maintaining infectivity, and/or maintaining viability of AAV particles comprises sodium phosphate, sodium chloride, cyclodextrin and a polysorbate. In certain embodiments, the cryoprotective formulation comprises an ionic strength from about 100 mM to about 700 mM. In certain embodiments, the cryoprotective formulation comprises an ionic strength from about 100 mM to about 400 mM. In certain embodiments, the cryoprotective formulation comprises an osmolality (mOsm/kg) from about 100 mOsm/kg to about 800 mOsm/kg. In certain embodiments, the cryoprotective formulation comprises an osmolality (mOsm/kg) from about 200 mOsm/kg to about 700 mOsm/kg. In certain embodiments, the sodium phosphate concentration at a concentration of about 1 mM to about 20 mM. In certain embodiments, the sodium chloride is at a concentration of about 100 mM to about 400 mM. In certain embodiments, the cyclodextrin comprises about 0.1% weight/volume (w/v) up to about 20% (w/v). In certain embodiments, the polysorbate comprises about 0.01% weight/volume (w/v) up to about 5% (w/v). In certain embodiments, the cryoprotective formulation is utilized for storing or maintaining viability of an AAV particle over periods of time at temperatures from about 20° C. to about −80° C. In certain embodiments, the cryoprotective formulation comprises a pH of about 7.0 to about 8.0. In certain embodiments, the AAV particle remains stable over multiple freeze/thaw cycles.

In another aspect, a cryoprotective formulation for storing, maintaining infectivity, and/or maintaining viability of AAV particles comprises a cryoprotective saccharide, a buffer, a salt and a non-ionic surfactant. In certain embodiments, the cryoprotective saccharide comprises cyclodextrin, trehalose or the combination thereof. In certain embodiments, the cryoprotective saccharide is trehalose. In certain embodiments, the trehalose is at a concentration of about 1% weight/volume (w/v) to about 20% w/v. In certain embodiments, the non-ionic surfactant is a polysorbate. In certain embodiments, the polysorbate is polysorbate 80. In certain embodiments, the polysorbate 80 is at a concentration of 0.001% w/v to about 1% w/v. In certain embodiments, the buffer is a tris buffer. In certain embodiments, the tris buffer is at a concentration of about 1 mM to about 20 mM. In certain embodiments, the salt is a magnesium salt. In certain embodiments, the magnesium salt is magnesium sulfate. In certain embodiments, the magnesium sulfate is at concentration of about 10 mM to about 250 mM. In certain embodiments, the cryoprotective formulation comprises an ionic strength from about 100 mM to about 700 mM. In certain embodiments, the cryoprotective formulation comprises an ionic strength from about 200 mM to about 500 mM. In certain embodiments, the cryoprotective formulation comprises an osmolality (mOsm/kg) from about 100 mOsm/kg to about 800 mOsm/kg. In certain embodiments, the cryoprotective formulation comprises an osmolality (mOsm/kg) from about 200 mOsm/kg to about 600 mOsm/kg. In certain embodiments, the cryoprotective formulation comprises a pH of about 7.0 to about 8.0. In certain embodiments, the cryoprotective formulation further comprises one or more pharmaceutical agents, cell culture media, proteins, lipids or combinations thereof. In certain embodiments, the formulation is utilized for storing or maintaining viability of an AAV particle over periods of time at temperatures from about 20° C. to about −80° C. In certain embodiments, the AAV particle remains stable over multiple freeze/thaw cycles.

In another aspect, a composition for storing, maintaining infectivity, and/or maintaining viability of AAV particles comprises sodium phosphate from about 1 mM to about 20 mM, sodium chloride from about 100 mM to about 400 mM, cyclodextrin from about 0.1% weight/volume (w/v) up to about 30% (w/v), and polysorbate from about 0.01% weight/volume (w/v) up to about 5% (w/v). In certain embodiments, the composition is utilized for storing or maintaining viability of an AAV particle over periods of time at temperatures from about 20° C. to about −80° C. In certain embodiments, the AAV particle remains stable over multiple freeze/thaw cycles.

In another aspect, a composition for storing, maintaining infectivity, and/or maintaining viability of AAV particles comprises trehalose from about 1% weight/volume (w/v) to about 20% w/v, polysorbate from 0.001% w/v to about 1% w/v, tris buffer from about 1 mM to about 20 mM, magnesium sulfate from about 10 mM to about 250 mM, cyclodextrin from about 0.1% weight/volume (w/v) up to about 30% (w/v), and polysorbate from about 0.01% weight/volume (w/v) up to about 5% (w/v). In certain embodiments, the composition is utilized for storing or maintaining viability of an adeno-associated virus (AAV), recombinant AAV (rAAV) particle, adenovirus particle or other viral particles over periods of time at temperatures from about 20° C. to about −80° C. In certain embodiments, the viral particle remains stable over multiple freeze/thaw cycles.

In another aspect, a formulation for storing or maintaining viability of an AAV, rAAV particle, adenovirus particle or other viral particle, at temperatures from ambient temperature or from about 20° C. to about −80° C. comprises about 1 mM to about 20 mM sodium phosphate, about 80 mM to about 300 mM sodium chloride (NaCl), about 0.1% to about 10% cyclodextrin, about 0.001% to about 5% polysorbate 80, wherein the formulation comprises an ionic strength from about 100 mM to about 500 mM and an osmolality of about 150 mOsm/kg to about 600 mOsm/kg.

In certain embodiments, the formulation is utilized for storing or maintaining viability of AAV particle over periods of time at temperatures from about 20° C. to about −80° C. In certain embodiments, the AAV particles are stable at about 4° C. In certain embodiments, the AAV particles are stable for at least six months to at least one year at 4° C. In certain embodiments, the AAV particle is stable over multiple freeze-thawing cycles. In certain embodiments, the AAV particle is stable over at least 5 freeze-thawing cycles.

In certain embodiments, the formulation comprises a buffer composition provided in Tables 1 and 2. In certain embodiments, the cryoprotective formulation comprises a buffer composition provided in Tables 1 or 2. In certain embodiments, the pharmaceutical composition comprises a buffer composition provided in Tables 1 or 2.

In one aspect, provided is a cryoprotective formulation for storing, maintaining infectivity, and/or maintaining viability of adeno-associated virus (AAV) particles, the formulation comprising a saccharide, a salt, a buffer, a surfactant, or combinations thereof.

In one aspect, provided is a pharmaceutical composition for storing, maintaining infectivity, and/or maintaining viability of AAV particles comprising a saccharide, a salt, a buffer, a surfactant, or combinations thereof.

In embodiments, the saccharide comprises one or more saccharides. In embodiments, the one or more saccharides comprise trehalose, cyclodextrin, sucrose, or combinations thereof. In embodiments, the saccharide is cyclodextrin. In embodiments, the cyclodextrin is at a concentration of about 0.1% weight/volume (w/v) up to about 20% (w/v). In embodiments, the cyclodextrin is at a concentration of about 0.1% (w/v) up to about 1% (w/v). In embodiments, the cyclodextrin is at a concentration of about 0.4% (w/v).

In embodiments, the saccharide is trehalose. In embodiments, the trehalose is at a concentration of about 0.5% (w/v) to about 20% (w/v). In embodiments, the trehalose is at a concentration of about 0.5% (w/v) to about 5% (w/v). In embodiments, the trehalose is at a concentration of about 1.1% (w/v).

In embodiments, the salt comprises a sodium salt, a magnesium salt, a calcium salt, a potassium salt, a phosphate salt, a sulfate salt, triethylamine, guanidine, N-substituted guanidine salts, acetamidine, N-substituted acetamidine, pyridine, picoline, ethanolamine, triethanolamine, dicyclohexylamine or N,N′-dibenzylethylenediamine salts, or combinations thereof. In embodiments, the salt is a sodium salt, a magnesium salt, a calcium salt, a potassium salt, a phosphate salt, a sulfate salt or combinations thereof. In embodiments, the salt is a sodium salt. In embodiments, the sodium salt is sodium chloride, sodium phosphate, or both. In embodiments, the sodium chloride is at a concentration of about 100 mM to about 400 mM. In embodiments, the sodium chloride is at a concentration of about 200 mM to about 350 mM. In embodiments, the sodium chloride is at a concentration of about 280 mM. In embodiments, the salt is a magnesium salt. In embodiments, the magnesium salt is magnesium sulfate. In embodiments, the magnesium sulfate is at concentration of about 10 mM to about 250 mM. In embodiments, the magnesium sulfate is at concentration of about 50 mM to about 200 mM. In embodiments, the magnesium sulfate is at concentration of about 125 mM.

In embodiments, the buffer comprises phosphate buffered saline (PBS), sodium phosphate, citric acid, acetic acid, tromethamine, aspartic acid, glutamic acid, HEPES, Tris, Bicine, acetate, glutamate, lactate, maleate, tartrate, phosphate, citrate, carbonate, glycinate, histidine, glycine, lysine, arginine, succinate, HEPES (4-(2-hydroxyethyl)-1-piperazineethanesulfonic acid), MOPS (3-(N-morpholino) propanesulfonic acid), MES (2-(N-morpholino)ethanesulfonic acid), triethanolamine buffer, or combinations thereof. In embodiments, the buffer comprises sodium phosphate. In embodiments, the sodium phosphate is at a concentration of about 1 mM to about 20 mM. In embodiments, the sodium phosphate is at a concentration of about 10 mM. In embodiments, the buffer comprises Tris. In embodiments, the Tris is at a concentration of about 1 mM to about 20 mM. In embodiments, the Tris is at a concentration of about 10 mM.

In embodiments, the surfactant is a non-ionic surfactant. In embodiments, the non-ionic surfactant comprises a polysorbate. In embodiments, the polysorbate is polysorbate 80. In embodiments, the polysorbate is at a concentration of about 0.01% (w/v) to about 5% (w/v). In embodiments, the polysorbate is at a concentration of 0.001% (w/v) to about 1% (w/v). In embodiments, the polysorbate is at a concentration of 0.02% (w/v). In embodiments, the cryoprotective formulation or pharmaceutical composition has an ionic strength of about 100 mM to about 700 mM. In embodiments, the cryoprotective formulation or pharmaceutical composition has an ionic strength of about 200 mM to about 600 mM. In embodiments, the cryoprotective formulation or pharmaceutical composition has an ionic strength of about 300 mM to about 500 mM. In embodiments, the cryoprotective formulation or pharmaceutical composition has an ionic strength of about 300 mM. In embodiments, the cryoprotective formulation or pharmaceutical composition has an ionic strength of about 500 mM.

In embodiments, the cryoprotective formulation or pharmaceutical composition has an osmolality from about 100 mOsm/kg to about 800 mOsm/kg. In embodiments, the cryoprotective formulation or pharmaceutical composition has an osmolality from about 200 mOsm/kg to about 600 mOsm/kg. In embodiments, the cryoprotective formulation or pharmaceutical composition has an osmolality of less than about 400 mOsm/kg. In embodiments, the cryoprotective formulation or pharmaceutical composition has an osmolality of about 200 mOsm/kg. In embodiments, the cryoprotective formulation or pharmaceutical composition has an osmolality of about 350 mOsm/kg.

In embodiments, the cryoprotective formulation or pharmaceutical composition has a pH of about 7.0 to about 8.0. In embodiments, the cryoprotective formulation or pharmaceutical composition has a pH of about 7.5.

In embodiments, the cryoprotective formulation or pharmaceutical composition comprises (1) cyclodextrin from about 0.1% (w/v) up to about 20% (w/v), (2) sodium chloride from about 100 mM to about 400 mM, (3) sodium phosphate from about 1 mM to about 20 mM, and (4) polysorbate from 0.001% (w/v) to about 1% (w/v). In embodiments, the cryoprotective formulation or pharmaceutical composition comprises (1) cyclodextrin at about 0.4% (w/v), (2) sodium chloride at about 280 mM, (3) sodium phosphate at about 10 mM, and (4) polysorbate 80 at about 0.02% (w/v). In embodiments, the cryoprotective formulation or pharmaceutical composition comprises (1) trehalose from about 1% (w/v) to about 20% (w/v), (2) magnesium sulfate from about 10 mM to about 250 mM, (3) Tris from about 1 mM to about 20 mM, and (4) polysorbate from 0.001% (w/v) to about 1% (w/v). In embodiments, the cryoprotective formulation or pharmaceutical composition comprises (1) trehalose at about 1.1% (w/v), (2) magnesium sulfate at about 125 mM, (3) Tris at about 10 mM, and (4) polysorbate 80 at about 0.02% (w/v).

In embodiments, the cryoprotective formulation or the pharmaceutical composition further comprises one or more of a pharmaceutical agent, medium, protein, or combinations thereof.

In embodiments, the AAV particles are derived from an AAV serotype selected from serotypes 1, 2, 3, 4, 5, 6, 7, 8, 9, 10, 11, 12, 13, 14, 15, 16, DJ or DJ/8. In embodiments, the AAV particles comprise a genome derived from AAV serotype 2. In embodiments, the AAV particles are comprise a capsid derived from AAV serotype 2.

In one aspect, provided is a method of storing or maintaining AAV particle viability and/or AAV particle infectivity at various temperatures, the method comprising depositing the AAV particle into a cryoprotective formulation disclosed herein or into a pharmaceutical composition disclosed herein. In embodiments, the AAV particle is derived from an AAV serotype selected from serotypes 1, 2, 3, 4, 5, 6, 7, 8, 9, 10, 11, 12, 13, 14, 15, 16, DJ or DJ/8. In embodiments, the AAV particle is comprises a genome derived from AAV serotype 2. In embodiments, the AAV particle comprises a capsid derived from AAV serotype 2. In embodiments, the AAV particle is stable over multiple freeze-thawing cycles. In embodiments, the AAV particle is stable over at least 5 freeze-thawing cycles. In embodiments, the AAV particle is stable at temperatures from about +20° C. to about −80° C. In embodiments, the AAV particle is stable at a temperature of about +4° C. In embodiments, the AAV particle is stable for at least six months at +4° C. In embodiments, the AAV particle is stable for at least one year at +4° C.

### BRIEF DESCRIPTION OF THE DRAWINGS

FIGS. 1A-1B are graphs demonstrating changes in osmolality with increasing concentration of cyclodextrin. FIG. 1A represents buffers possessing 200 mM ionic strength and FIG. 1B represents buffers with the ionic strength of 300 mM. A line was fitted through the three data points and an equation was generated. Osmolality was measured with an osmometer, n=1.

FIGS. 2A-2C are a series of graphs showing changes in osmolality with increasing concentration of trehalose. FIG. 2A represents buffers possessing 200 mM ionic strength. FIG. 2B represents buffers with the ionic strength of 350 mM. FIG. 2C illustrates buffers with the ionic strength of 500 mM. A line was fitted through the three data points and an equation was generated. Osmolality was measured with an osmometer, n=1.

FIG. 3 is a series of graphs demonstrating AAV2 stability in different buffers from Formulation A and storage conditions over time. Changes to VP (viral particle) titer, VG (vector genome) titer, monomer area and higher molecular weight species (HMW) were monitored. Samples were thawed at the same time for at least 1 hour at room temperature. The sample analysis consisted of the following techniques or instruments: Gyrolab for VP titer, qPCR for VG titer, HPLC-SEC (high-performance liquid chromatography-size exclusion chromatography) for both monomer area and HMW content. Variation amongst replicates for VP titer was below 5%, whilst that for VG titer was below 20%. The starting AAV2 VG titer was 1.2×10¹² VG/mL for all buffers. The graphs contain a confidence region for the fitted line that illustrates the predicted trend of the data over time. See Table 1 for the buffer compositions that belong to Formulation A.

FIGS. 4A-4B are a series of graphs demonstrating the relative standard deviation (RSD) of the total VP and HMW data for the two formulations. FIG. 4A illustrates the VP and HMW data for buffer 3 for Formulation A (See Table 1). FIG. 4B depicts the VP and HMW data for buffer 7 for Formulation B (See Table 2). All storage conditions were used to calculate RSD value in percentage.

FIGS. 5A-5B are plots demonstrating AAV2 stability in the two chosen buffers with a 10-fold lower product concentration. FIG. 5A shows data for buffer 3 for Formulation A and FIG. 5B shows data for buffer 7 for Formulation B (See Tables 1 and 2). Changes to VP titer, monomer area and higher molecular weight species (HMW) for the two formulations were monitored. Samples were thawed at the same time for at least 1 hour at room temperature. Analysis that was performed consisted of the following techniques or instruments: Gyrolab for VP titer, HPLC-SEC for both monomer area and HMW content. Variation amongst replicates for VP titer was below 5%. The starting AAV2 VG titer was 1.0×10¹¹ VG/mL for all buffers. The graphs contain a confidence region for the fitted line that illustrates the predicted trend of the data over time. Monomer area result for the Formulation A—Buffer 3 for 6^{th} freeze/thaw cycle at −80° C. is 1802 mAU.

FIG. 6 is a graph showing a summary of the earlier stability data using relative standard deviation. All storage conditions were used to calculate RSD value in percentage. Formulation A-3 is buffer 3 for Formulation A and Formulation B-7 is buffer 7 for Formulation B.

FIG. 7 is a graph demonstrating the ratio of infectious particles to vector genomes before and after their exposure to different storage conditions. Control is the sample frozen at −80° C. on the day of its generation and defrosted only once. Error bars represent ±1 SD that was generated by propagating the error of the VG titer assay and the infectious titer assay.

### DETAILED DESCRIPTION

It is demonstrated herein that an appropriate selection of excipients, and the balance of ionic strength and osmolality leads to reproducible and stable product quality of viruses, such as an AAV particle, at, for example, room temperature or at 4° C., and/or provides the ability to withstand at least 10 freeze/thaw events. The embodiments provided herein reduce reliance on the −80° C. cold chain and as such allow for improved manufacturability and distribution, as well as alternative product containment options that can be implemented for improved product accessibility.

Adeno-associated viruses (AAV) are small, single-stranded DNA viruses which require helper virus to facilitate efficient replication. The 4.7 kb genome of AAV is characterized by two inverted terminal repeats (ITR) and two open reading frames which encode the Rep proteins and Cap proteins, respectively. The rep reading frame encodes four proteins of molecular weight 78 kD, 68 kD, 52 kD, and 40 kD. These proteins function mainly in regulating AAV replication and rescue and integration of the AAV into a host cell's chromosomes. The cap reading frame encodes three structural proteins of molecular weight 85 kD (VP 1), 72 kD (VP2), and 61 kD (VP3), which form the virion capsid. More than 80% of the total proteins in an AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

The AAV particles described herein are not limited to a specific serotype, and any AAV serotype, as well as AAV variants, are suitable with the pharmaceutical compositions described herein.

In some embodiments, the AAV is an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh.10 capsid proteins).

In some embodiments, the AAV a pseudotyped AAV, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins). In some embodiments, the pseudotyped AAV is AAV2/7m8 (i.e., an AAV containing AAV2 ITRs and AAV7m8 capsid proteins). In some embodiments, the pseudotyped AAV is AAV2/8 (i.e., an AAV containing AAV2 ITRs and AAV capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In embodiments, the capsid is a variant AAV capsid such as the AAV2 variant rAAV2-retro (SEQ ID NO:44 from WO 2017/218842, incorporated herein by reference).

In some embodiments, the AAV contains two or more capsid proteins selected from different serotypes. In some embodiments, the AAV contains an rAAV2-retro and an AAVrh.10 capsid protein.

In embodiments, the AAV genome and/or the AAV capsid are selected from serotypes 1, 2, 3, 4, 5, 6, 7, 8, 9, 10, 11, 12, 13, 14, 15, 16, DJ or DJ/8.

In certain embodiments, the AAV particle comprises a genome derived from AAV serotype 2. In certain embodiments, the AAV particle comprises a capsid derived from AAV serotype 2.

The AAV particles described can be used in any downstream application that is compatible with AAV particles. For example, in some embodiments, the AAV particles are suitable for use in gene therapy. In some embodiments, the purified AAV particles comprise a therapeutic gene. However, it is also understood that the AAV particles can also be useful as a vaccine. Further, the downstream applications can extend beyond therapeutic use. For example, the AAV particles describe herein can also be useful for imaging applications, or other non-therapeutic uses. In some embodiments, the AAV particles contain an encapsidated recombinant vector sequence. In some embodiments, it may be desirable to have AAV particles that include empty capsids; AAV particles that include a mixture of full capsids and empty capsids; AAV particles that include a mixture of full capsids, partially-full capsids, and empty capsids; or AAV particles that include a mixture of partially-full capsids and empty capsids. In some embodiments, the formulations and pharmaceutical compositions disclosed herein comprise AAV particles that have empty capsids. In some embodiments, the formulations and pharmaceutical compositions disclosed herein comprise AAV particles that have full capsids, partially fully capsids, or a mixture of full capsids and empty capsids. In some embodiments, the formulations and pharmaceutical compositions disclosed herein comprise AAV particles that have a mixture of full capsids, partially-full capsids, and empty capsids. In some embodiments, the AAV particles have a mixture of partially-full capsids and empty capsids.

A recombinant AAV—“rAAV”—includes any AAV derived from any adeno-associated virus serotype. rAAVs can have one or more of the AAV wild-type genes deleted in whole or in part, preferably the rep and/or cap genes, but retain functional flanking ITR sequences.

As used herein, a “vector” is a vehicle that comprises a polynucleotide to be delivered into a host cell, either in vitro or in vivo. The term includes, as examples, plasmids, expression vectors, viral vectors and viruses. In embodiments, a vector refers to a virion comprising a recombinant viral genome (e.g., rAAV), wherein the viral genome comprises one or more ITRs and a transgene.

As used herein, the terms “AAV particle”, “rAAV particle”, “virus particle,” and “AAV virion” are intended to mean genome-containing (also known as “full capsids” or “partially-full capsids”), as well as empty capsids, or any combination of full, partially-full, and empty capsids, unless specified otherwise.

“Empty capsids” and “empty particles” refer to AAV particles (e.g., rAAV particles) having an AAV capsid, but lacking in whole, or in part, an AAV genome (e.g., recombinant AAV genome comprising a transgene sequence and one or two ITRs). Such empty capsids do not function to transfer a transgene into a target cell or cells.

As used herein, the term “titer” is intended to mean the quantity of virus in a given volume. A viral titer can include a “physical titer” or a “functional titer.” The physical titer is a measurement of how much virus is present and is generally expressed as the number of viral particles per mL (VP/mL), or vector genomes per mL (VG/mL) which is interchangeable with genome copies per mL (GC/mL). Functional titer, or infectious titer, is the measurement of how much virus actually infects a target cell and is generally expressed in the form of transduction units per mL (TU/mL), or for adenovirus as plaque-forming units per mL (pfu/mL) or infectious units per mL (ifu/mL). It is understood that functional titer will generally be lower than physical titer, usually by a factor of about 10 to about 100-fold.

In various embodiments, the virus is a chimeric virus, a synthetic virus, a recombinant virus, a mosaic virus or a pseudotyped virus.

As used herein, the term “freeze-thawing cycles,” “freeze/thaw cycles,” or “freeze-thaw cycles” refers to when the temperature changes from above freezing, to below freezing, and then returns to above freezing.

Ranges: throughout this disclosure, various aspects of the disclosure can be presented in a range format. It should be understood that a description of numeric values in a range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, preferably within 5-fold, and also preferably within 2-fold, of a value. Where particular values are described in the application and claims, unless otherwise stated, the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

Any formulations, pharmaceutical compositions or methods provided herein can be combined with one or more of any of the other formulations, pharmaceutical compositions and methods provided herein.

The terminology used herein is for the purpose of describing particular embodiments only and is not intended to be limiting of the invention. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.” The transitional term “comprising,” which is synonymous with “including,” “containing,” or “characterized by,” is inclusive or open-ended and does not exclude additional, unrecited elements or method steps. When used herein the term “comprising” can be substituted with the term “containing” or “including” or sometimes when used herein with the term “having.” By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

As used in this specification and the appended claims, the term “or” is generally employed in its sense including “and/or” unless the content clearly dictates otherwise.

As used herein, the terms “and/or” means any one of the items, any combination of the items, or all of the items with which this term is associated.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Cryoprotectants

In certain embodiments, the formulations and pharmaceutical compositions disclosed herein comprise one or more cryoprotectants. As used herein, the terms “cryoprotectant” or “cryoprotective” refer to a chemical or a chemical solution which facilitates the process of cryoprotection by reducing the injury of the stored components (e.g., viral particles, virus, cells and/or tissues) during freezing and thawing, or reduces injury of the stored components at room temperature at about +20° C. or at about +4° C. A cryoprotective formulation or a cryoprotective composition protects the stored components (i.e., AAV particles) from damage associated with storage at sub-zero temperatures and/or freezing, e.g., viral capsid or cell membrane damage due to ice crystal formation, or at room temperature at about +20° C. or at about +4° C. In some embodiments, multiple different cryoprotectants can be combined.

In some embodiments, the cryoprotectant is a saccharide. In some embodiments, the cryoprotectant is a saccharide.

In certain embodiments, the saccharides comprise trehalose, cyclodextrin, sucrose or combinations thereof. In certain embodiments, the saccharide is trehalose. In certain embodiments, the saccharide is cyclodextrin.

As used herein, the term “saccharide” refers to any carbohydrate comprising monosaccharides (e.g., glucose, ribose, fructose, galactose, etc.), disaccharides (e.g., sucrose, lactose, maltose, cellobiose, trehalose, dextran e.g., dextran-40, melibiose, etc.), oligosaccharides (e.g., raffinose, stachyose, amylose, etc.), and polysaccharides (e.g., starch, glycogen, cellulose, chitin, xylan, arabinoxylan, mannan, fucoidan, galactomannan, callose, laminarin, chrysolaminarin, amylopectin, dextran, dextrins, maltodextrins, inulin, oligofructose, polydextrose, etc.). The term encompasses simple carbohydrates, as well as complex carbohydrates. Indeed, it is not intended that the present disclosure be limited to any particular saccharide, as various saccharides and forms of saccharides find use in the present disclosure.

In certain aspects, the cryoprotective formulation for storing, maintaining infectivity, and/or maintaining viability of AAV particles comprises sodium phosphate, sodium chloride, cyclodextrin and a polysorbate.

In certain aspects, the cryoprotective formulation for storing, maintaining infectivity, and/or maintaining viability of AAV particles comprises a cryoprotective saccharide, a buffer, a salt and a non-ionic surfactant.

In certain aspects, the cryoprotective formulation comprises a buffer composition provided in Tables 1 or 2.

In certain embodiments, the formulations or pharmaceutical compositions embodied herein, comprise a saccharide from about 0.001% w/v to about 30% w/v, about 0.005% w/v to about 25% w/v, about 0.01% w/v to about 24% w/v, about 0.05% w/v to about 23%, about 0.1% to about 22% w/v, 0.1% w/v to about 21% w/v, 0.1% w/v to about 20% w/v, about 0.5% w/v to about 19% w/v, about 1% w/v to about 18% w/v, about 1% w/v to about 17% or about 1% to about 16% w/v.

In certain embodiments, the formulations or pharmaceutical compositions embodied herein, comprise cyclodextrin from about 0.001% w/v to about 30% w/v, about 0.005% w/v to about 25% w/v, about 0.01% w/v to about 24% w/v, about 0.05% w/v to about 23% or about 0.1% to about 22% w/v. In certain embodiments, the formulations or pharmaceutical compositions embodied herein, comprise about 0.375% w/v cyclodextrin.

In certain embodiments, the formulations or pharmaceutical compositions embodied herein, comprise trehalose from about 0.1% w/v to about 20% w/v, about 0.5% w/v to about 19% w/v, about 1% w/v to about 18% w/v, about 1% w/v to about 17% or about 1% to about 16% w/v. In certain embodiments, the formulations or pharmaceutical compositions embodied herein, comprise about 1.1% w/v trehalose.

### Saccharides and Other Cryoprotectants

In some embodiments, the formulations or pharmaceutical compositions embodied herein comprise one or more cryoprotectants, wherein the cryoprotectant is a solvent (e.g., an organic solvent), a polyol, a polymer, a saccharide, or a combination thereof. In some embodiments, the cryoprotectant is DMSO (dimethyl sulfoxide), ethylene glycol, glycerol, propylene glycol, 2-methyl-2,4-pentanediol (MP), glycerol-3-phosphate, diethyl glycol, triethylene glycol, a polyvynyl alcohol, PEG, hydroxyethyl starch, sorbitol, mannitol, lactose, sucrose, trehalose, or a combination thereof. In some embodiments, the cryoprotectant is an organic solvent. In some embodiments, the cryoprotectant is a polyol. In some embodiments, the cryoprotectant is a polymer. In some embodiments, the cryoprotectant is DMSO (dimethyl sulfoxide). In some embodiments, the cryoprotectant is ethylene glycol. In some embodiments, the cryoprotectant is glycerol. In some embodiments, the cryoprotectant is propylene glycol. In some embodiments, the cryoprotectant is (MPD) 2-methyl-2,4-pentanediol. In some embodiments, the cryoprotectant is glycerol-3-phosphate. In some embodiments, the cryoprotectant is diethyl glycol. In some embodiments, the cryoprotectant is triethylene glycol. In some embodiments, the cryoprotectant is a polyvynyl alcohol. In some embodiments, the cryoprotectant is PEG. In some embodiments, the cryoprotectant is hydroxyethyl starch. In some embodiments, the cryoprotectant is sorbitol. In some embodiments, the cryoprotectant is mannitol. In some embodiments, the cryoprotectant is lactose.

In embodiments, multiple (e.g., 2, 3, 4, 5, etc.) cryoprotectants can be combined.

### Solvents

In certain aspects, as described above, the formulations or pharmaceutical compositions may include one or more solvents. Any solvent suitable for stably maintaining the AAV particle may be incorporated into the compositions according to the present disclosure. Some non-limiting examples include Dulbecco's Modified Eagle Medium (DMEM), Eagle's Minimal Essential Medium (EMEM), X-VIVO, water, saline, dextrose, and combinations thereof. In certain embodiments, the compositions include DMEM. In certain embodiments, the compositions include EMEM.

### Pharmaceutically Acceptable Salts

In embodiments, the formulations and pharmaceutical compositions disclosed herein include one or more pharmaceutically acceptable salts. As used herein, the term “pharmaceutically acceptable salt” refers to a salt prepared from a pharmaceutically acceptable non-toxic acid or base including an inorganic acid and base and an organic acid and base. Suitable pharmaceutically acceptable base addition salts of the pharmaceutically acceptable compositions disclosed herein, include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts.

In certain embodiments, the pharmaceutically acceptable salt is a metal salt and salt of ammonia or organic amines that are safe for administration to a subject (e.g., a human) in a drug formulation. Suitable pharmaceutically acceptable salts include, but are not limited to, sodium, potassium, magnesium, calcium, cesium, ammonium, triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sultanates such as methanesulfonate, ethanesulfonate, benzenesulfonate, p-toluenesulfonate, camphor sultanate and naphthalenesulfonate; amino acid salts, such as arginate, alaninate, asparaginate and glutamate; and carbohydrate salts such as gluconate and galacturonate. Other salts that may be used herein are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995), which are incorporated herein by reference in their entirety for all intent and purposes. In some embodiments, the pharmaceutically acceptable salt is a sodium salt, a magnesium salt, a calcium salt, a potassium salt, a phosphate salt, or a sulfate salt. In some embodiments, the pharmaceutically acceptable salt is a metal salt.

In some embodiments, the pharmaceutically acceptable salt is a sodium salt. In some embodiments, the pharmaceutically acceptable salt is a magnesium salt. In some embodiments, the pharmaceutically acceptable salt is a calcium salt. In some embodiments, the pharmaceutically acceptable salt is a potassium salt. In some embodiments, the pharmaceutically acceptable salt is a phosphate salt. In some embodiments, the pharmaceutically acceptable salt is a sulfate salt. In certain embodiments, the sodium salt comprises sodium chloride, sodium phosphate or the combination thereof. In some embodiments, the pharmaceutically acceptable salt is sodium chloride. In some embodiments, the pharmaceutically acceptable salt is sodium phosphate. In some embodiments, the magnesium salt is magnesium sulfate.

In certain embodiments, the salt comprises a sodium salt, a magnesium salt, a calcium salt, a potassium salt, a phosphate salt, a sulfate salt, triethylamine, guanidine, N-substituted guanidine salts, acetamidine, N-substituted acetamidine, pyridine, picoline, ethanolamine, triethanolamine, dicyclohexylamine or N,N′-dibenzylethylenediamine salts, or combinations thereof. In certain embodiments, the sodium salt comprises sodium chloride, sodium phosphate, or the combination thereof. In certain embodiments, the magnesium salt is magnesium sulfate. In certain embodiments, the salt comprises sodium chloride, sodium phosphate, magnesium sulfate or the combination thereof.

In some embodiments, the pharmaceutical composition comprises a pharmaceutically acceptable salt concentration of about 1 mM to about 300 mM. In some embodiments, the pharmaceutically acceptable salt concentration is about 2 mM to about 295 mM. In some embodiments, the pharmaceutically acceptable salt concentration is about 3 mM to about 290 mM. In some embodiments, the pharmaceutically acceptable salt concentration is about 4 mM to about 285 mM. In some embodiments, the pharmaceutically acceptable salt concentration is about 6 mM to about 284 mM. In some embodiments, the pharmaceutically acceptable salt concentration is about 7 mM to about 283 mM. In some embodiments, the pharmaceutically acceptable salt concentration is about 8 mM to about 282 mM. In some embodiments, the pharmaceutically acceptable salt concentration is about 9 mM to about 281 mM. In some embodiments, the pharmaceutically acceptable salt concentration is about 10 mM to about 280 mM.

In some embodiments, the pharmaceutically acceptable salt is sodium chloride. In certain embodiments the sodium chloride concentration is about 280 mM.

In some embodiments, the pharmaceutically acceptable salt is sodium phosphate. In some embodiments, the sodium phosphate concentration is about 10 mM.

In certain embodiments, the formulations or pharmaceutical compositions comprise sodium phosphate at a concentration of about 10 mM and sodium chloride at a concentration of about 280 mM.

In certain embodiments, the pharmaceutically acceptable salt is magnesium sulfate. In certain embodiments, the magnesium sulfate is at a concentration of about 30 mM to about 200 mM, about 35 mM to about 190 mM, about 40 mM to about 180 mM, about 40 mM to about 170 mM, about 45 mM to about 160 mM, about 46 mM to about 150 mM, about 47 mM to about 140 mM, about 48 mM to about 130 mM, about 49 mM to about 128 mM, about 50 mM to about 125 mM. In certain embodiments, the magnesium sulfate is at a concentration of about 50.

In some aspects of the present disclosure, the formulations and the pharmaceutical compositions include a pharmaceutically acceptable salt, a buffering agent, a cryoprotectant, and a non-ionic surfactant.

In certain aspects, the formulations or pharmaceutical compositions may further include one or more pharmaceutically acceptable excipient or is diluted in a pharmaceutically acceptable excipient to obtain the desired ratio of agents in the compositions or formulations. A pharmaceutically acceptable excipient, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular formulation desired. Remington's The Science and Practice of Pharmacy, 21st Edition, A. R. Gennaro, (Lippincott, Williams & Wilkins, Baltimore, Md., 2006; incorporated herein by reference) discloses various excipients used in formulating pharmaceutical compositions which excipients are useful in preparing the present compositions. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this disclosure. In certain embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved for use in humans by the United States Food and Drug Administration (FDA). In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

In certain aspects, the formulations or pharmaceutical compositions may further include one or more pharmaceutically acceptable excipient or is diluted in a pharmaceutically acceptable excipient to obtain the desired ratio of agents in the compositions or formulations. A pharmaceutically acceptable excipient, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular formulation desired. Remington's The Science and Practice of Pharmacy, 21st Edition, A. R. Gennaro, (Lippincott, Williams & Wilkins, Baltimore, Md., 2006; incorporated herein by reference) discloses various excipients used in formulating pharmaceutical compositions which excipients are useful in preparing the present compositions. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this disclosure. In certain embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved for use in humans by the United States Food and Drug Administration (FDA). In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

### Non-Ionic Surfactants

In certain embodiments, the formulations or pharmaceutical compositions disclosed herein comprise one or more non-ionic surfactants. As used herein, the term “non-ionic surfactant” refers to surfactants that are composed of polar head groups that lack an electric charge. In some embodiments, the non-ionic surfactant is a copolymer. In some embodiments, the non-ionic surfactant is a poloxamer. A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename Pluronic®. Because the lengths of the polymer blocks can be customized, many different poloxamers exist that have slightly different properties. For the generic term “poloxamer,” these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). In some embodiments, the poloxamer is P188, P237, P338, or P407. In some embodiments, the poloxamer is P188. In some embodiments, the poloxamer is P237. In some embodiments, the poloxamer is P338. In some embodiments, the poloxamer is P407. In some embodiments the non-ionic surfactant is a polyoxyethylene sorbitan esters surfactant (commonly referred to as the Tweens), such as PS-20 and PS-80; a copolymer of ethylene oxide (EO), a phospholipid such as phosphatidyl choline (lecithin); a polyoxyethylene fatty ether derived from lauryl, cetyl, stearyl and oleyl alcohols (known as Brij surfactants), such as triethyleneglycol monolauryl ether (Brij 30) or polyoxyethylene (23) lauryl ether (Brij™ 35); and a sorbitan ester (commonly known as the SPANs), such as sorbitan trioleate (Span™ 85) and sorbitan monolaurate. In some embodiments, the non-ionic surfactant is polysorbate 20 (PS-20), polysorbate 80 (PS-80), or Brij surfactant, or a combination thereof. In some embodiments, the non-ionic surfactant is a polysorbate. In some embodiments, the polysorbate is PS-20. In some embodiments, the polysorbate is PS-40. In some embodiments, the polysorbate is PS-60. In some embodiments, the polysorbate is PS-80. In some embodiments, the non-ionic surfactant is a Brij surfactant. In some embodiments, the non-ionic surfactant is a copolymer of EO. In some embodiments, the non-ionic surfactant is a phospholipid. In some embodiments, the non-ionic surfactant is a phosphatidyl choline (lecithin). In some embodiments, the non-ionic surfactant is a polyoxyethylene fatty ether derived from a lauryl alcohol. In some embodiments, the non-ionic surfactant is a polyoxyethylene fatty ether derived from a cetyl alcohol. In some embodiments, the non-ionic surfactant is a polyoxyethylene fatty ether derived from a stearyl alcohol. In some embodiments, the non-ionic surfactant is a polyoxyethylene fatty ether derived from an oleyl alcohol. In some embodiments, the non-ionic surfactant is a triethyleneglycol monolauryl ether (Brij™ 30). In some embodiments, the non-ionic surfactant is a polyoxyethylene (23) lauryl ether (Brij™ 35). In some embodiments, the non-ionic surfactant is a sorbitan ester. In some embodiments, the non-ionic surfactant is a sorbitan trioleate (Span™ 85). In some embodiments, the non-ionic surfactant is a sorbitan monolaurate.

In some embodiments, the formulations or pharmaceutical compositions disclosed herein comprise non-ionic surfactant(s) from about 0.0001% (w/v) to about 0.1% (w/v), about 0.0005% (w/v) to about 0.005% (w/v), about 0.00075% (w/v) to about 0.0025% (w/v), about 0.0050% (w/v) to about 0.0075% (w/v), about 0.01% (w/v) to about 0.015% (w/v), about 0.0175% (w/v) to about 0.018% (w/v), about 0.019% (w/v) to about 0.02% (w/v), about 0.025% (w/v) to about 0.03% (w/v), or about 0.4% (w/v) to about 0.5% (w/v). In some embodiments, the formulations or pharmaceutical compositions disclosed herein comprise about 0.02% (w/v) non-ionic surfactant.

### Ionic Strength

As provided herein, the formulations or pharmaceutical compositions of the present disclosure can have an ionic strength of about 50 mM to about 800 mM. In certain embodiments, the ionic strength is about 75 mM to about 750 mM, about 100 mM to about 700 mM, about 150 mM to about 675 mM, about 175 mM to about 650 mM, about 180 mM to about 600 mM, about 190 mM to about 550 mM, or about 200 mM to about 500 mM. In certain embodiments, the formulations or pharmaceutical compositions described herein have an ionic strength of about 300 mM or about 500 mM.

### Osmolality

As provided herein, the formulations or pharmaceutical compositions of the present disclosure can have an osmolality of about 100 mOsm/kg to about 800 mOsm/kg. In certain embodiments, the osmolality is about 150 mOsm/kg to about 750 mOsm/kg, about 175 mOsm/kg to about 700 mOsm/kg, about 180 mOsm/kg to about 675 mOsm/kg, about 185 mOsm/kg to about 650 mOsm/kg, or about 190 mOsm/kg to about 628 mOsm/kg. In certain embodiments, the osmolality is about 200 mOsm/kg, or about 350 mOsm/kg, or about 500 mOsm/kg, or about 600 mOsm/kg. In certain embodiments, the formulations or pharmaceutical compositions of the present disclosure have an osmolality of about 200 mOsm/kg or about 350 mOsm/kg. In certain embodiments, the formulations or pharmaceutical compositions of the present disclosure have an osmolality of about 200 mOsm/kg or about 600 mOsm/kg. In certain embodiments, the formulations or pharmaceutical compositions of the present disclosure have an osmolality of less than about 400 mOsm/kg.

### pH Conditions

As provided herein, the formulations or pharmaceutical compositions of the present disclosure can have a pH that is about 4.0 to about 9.0. In some embodiments, the pH of the formulations or pharmaceutical compositions is about 7.0 to about 8.0. In certain embodiments, the pH of the formulations or pharmaceutical compositions is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some embodiments the pH of the formulations or pharmaceutical compositions of the present disclosure is about 7.5.

### Formulations and Pharmaceutical Composition

The formulations or pharmaceutical compositions provided herein can be either a liquid composition or a frozen composition. Accordingly, in some embodiments, the formulations or pharmaceutical compositions are in a liquid state. In other embodiments, the formulations or pharmaceutical compositions are in a solid or semi-solid state.

In certain aspects, the formulation for storing, maintaining infectivity, and/or maintaining viability of AAV particles, comprises a saccharide, a buffer, a surfactant, a salt or combinations thereof. In certain aspects, the pharmaceutical composition for storing, maintaining infectivity, and/or maintaining viability of AAV particles comprises a cryoprotectant, a buffer, a surfactant, and a salt.

In certain aspects, the composition for storing, maintaining infectivity, and/or maintaining viability of AAV particles comprises sodium phosphate from about 1 mM to about 20 mM, sodium chloride from about 100 mM to about 400 mM, cyclodextrin from about 0.1% weight/volume (w/v) up to about 20% (w/v), and polysorbate from about 0.01% weight/volume (w/v) up to about 5% (w/v).

In certain aspects, the composition for storing, maintaining infectivity, and/or maintaining viability of AAV particles comprises trehalose from about 1% weight/volume (w/v) to about 20% w/v, polysorbate from 0.001% w/v to about 1% w/v, tris buffer from about 1 mM to about 20 mM, magnesium sulfate from about 10 mM to about 250 mM, cyclodextrin from about 0.1% weight/volume (w/v) up to about 20% (w/v), and polysorbate from about 0.01% weight/volume (w/v) up to about 5% (w/v).

In certain aspects, the pharmaceutical composition comprising a buffer composition provided in Tables 1 or 2.

In certain aspects, the formulation comprises a buffer composition provided in Tables 1 or 2.

In certain aspects, the formulation comprises about 5 mM to about 15 mM sodium phosphate, about 150 mM to about 200 mM NaCl, about 1% to about 2% cyclodextrin, about 0.01% to about 0.5% polysorbate 80, wherein the formulation comprises an ionic strength from about 180 mM to about 250 mM, and an osmolality from about 300 mOsm/kg to about 400 mOsm/kg.

In certain aspects, the formulation comprises about 10 mM sodium phosphate, about 180 mM NaCl, about 1.5% cyclodextrin, about 0.1% polysorbate 80, wherein the formulation comprises an ionic strength of about 206 mM and an osmolality of about 367 mOsm/kg.

In certain aspects, the formulation comprises about 10 mM sodium phosphate, about 180 mM NaCl, about 0.375% cyclodextrin, about 0.02% polysorbate 80, wherein the formulation comprises an ionic strength of about 200 mM and an osmolality of about 350 mOsm/kg.

In certain aspects, the formulation comprises about 10 mM sodium phosphate, about 180 mM NaCl, about 21.0% cyclodextrin, about 0.02% polysorbate 80, wherein the formulation comprises an ionic strength of about 200 mM and an osmolality of about 600 mOsm/kg.

In certain aspects, the formulation comprises about 10 mM sodium phosphate, about 280 mM NaCl, about 0.375% cyclodextrin, about 0.02% polysorbate 80, wherein the formulation comprises an ionic strength of about 300 mM and an osmolality of about 350 mOsm/kg.

In certain aspects, the formulation comprises about 10 mM sodium phosphate, about 280 mM NaCl, about 11.5% cyclodextrin, about 0.02% polysorbate 80, wherein the formulation comprises an ionic strength of about 300 mM and an osmolality of about 600 mOsm/kg.

In certain aspects, the formulation comprises about 5 mM to about 20 mM tris, about 30 mM to about 80 mM MgSO₄, about 0.5% to about 3% trehalose, about 0.01% to about 0.5% polysorbate 80, wherein the formulation comprises an ionic strength from about 180 mM to about 250 mM, and an osmolality from about 100 mOsm/kg to about 200 mOsm/kg.

In certain aspects, the formulation comprises about 5 mM to about 15 mM tris, about 40 mM to about 70 mM MgSO₄, about 1% to about 2% trehalose, about 0.05% to about 0.5% polysorbate 80, wherein the formulation comprises an ionic strength from about 200 mM to about 240 mM, and an osmolality from about 110 mOsm/kg to about 140 mOsm/kg.

In certain aspects, the formulation comprises about 10 mM tris, about 125 MgSO₄, about 1.1% trehalose, about 0.02% polysorbate 80, wherein the formulation comprises an ionic strength of about 500 mM, and an osmolality of about 200 mOsm/kg.

In certain aspects, the formulation comprises about 10 mM tris, about 55 mM MgSO₄, about 1.5% trehalose, about 0.1% polysorbate 80, wherein the formulation comprises an ionic strength of about 220 mM, and an osmolality of about 128 mOsm/kg.

### Storage Conditions

In certain embodiments, the formulations or pharmaceutical compositions disclosed herein are stored at ambient or room temperature, such as about 25° C. In some embodiments, the compositions disclosed herein are stored at less than about 25° C. In some embodiments, the compositions disclosed herein are stored between about 0° C. to about 25° C. In some embodiments, the compositions are stored between about 0° C. to about 10° C. In some embodiments, the compositions are stored between about 2° C. to about 8° C. In some embodiments, the compositions are stored at about 4° C. In some embodiments, the compositions are stored below 0° C. In some embodiments, the compositions are stored between about −20° C. to about −80° C. In some embodiments, the compositions are stored at about −20° C. In some embodiments, the compositions are stored at about −70° C. In some embodiments, the compositions are stored at about −80° C. In certain embodiments, the compositions are stored at or below 0° C., and the stability is maintained or enhanced after one or more freeze/thaw cycles. In certain embodiments, the compositions are stored at about −20° C., and the stability is maintained or enhanced after one or more freeze/thaw cycles. In certain embodiments, the compositions are stored at about −20° C., and the stability is maintained or enhanced after one or more freeze/thaw cycles. In certain embodiments, the compositions are stored at about −70° C., and the stability is maintained or enhanced after one or more freeze/thaw cycles. In certain embodiments, the compositions are stored at about −80° C., and the stability is maintained or enhanced after one or more freeze/thaw cycles. In some embodiments, the stability is maintained after one freeze/thaw cycle. In some embodiments, the stability is maintained after more than one freeze/thaw cycle. In some embodiments, the stability is enhanced. In some embodiments, the stability is enhanced after one freeze/thaw cycle. In some embodiments, the stability is enhanced after more than one freeze/thaw cycle.

The formulations or pharmaceutical compositions disclosed herein can maintain or enhance the stability of the AAV particles and/or decrease or prevent the aggregation after one of more freeze/thaw cycles. In some embodiments, the compositions disclosed herein maintain the stability of the AAV particles. In some embodiments, the compositions disclosed herein decrease the aggregation after one freeze/thaw cycle. In some embodiments, the compositions disclosed herein decrease the aggregation after more than one freeze/thaw cycle. In some embodiments, the compositions disclosed herein prevents the aggregation after one freeze/thaw cycle. In some embodiments, the compositions disclosed herein prevents the aggregation after more than one freeze/thaw cycle.

In some embodiments, the stability is maintained or enhanced after two or more freeze/thaw cycles. In some embodiments, the stability is maintained or enhanced after three or more freeze/thaw cycles. In some embodiments, the stability is maintained or enhanced after four or more freeze/thaw cycles. In some embodiments, the stability is maintained or enhanced after four or more freeze/thaw cycles. In some embodiments, the stability is maintained or enhanced after five or more freeze/thaw cycles. In some embodiments, the stability is maintained or enhanced after six or more freeze/thaw cycles. In some embodiments, the stability is maintained or enhanced after seven or more freeze/thaw cycles. In some embodiments, the stability is maintained or enhanced after eight or more freeze/thaw cycles. In some embodiments, the stability is maintained or enhanced after nine or more freeze/thaw cycles. In some embodiments, the stability is maintained or enhanced after ten or more freeze/thaw cycles.

In another aspect, a method of storing or maintaining viability of an AAV particle, comprising depositing the AAV particle in a formulation or pharmaceutical composition embodied herein. In certain embodiments, the formulation is utilized for storing or maintaining viability of AAV particle over periods of time at temperatures from about 20° C. to about −80° C. In certain embodiments, the AAV particle remains stable over multiple freeze/thaw cycles. In certain embodiments, the AAV particle is stable over multiple freeze-thawing cycles. In certain embodiments, the AAV particle is stable over at least 5 multiple freeze-thawing cycles. In certain embodiments, the AAV particle is stable at temperatures from about 20° C. to about −80° C. In certain embodiments, the AAV particle is stable at about 4° C. In certain embodiments, the AAV particle is stable for at least six months to at least one year.

In another aspect, a method of cryopreserving a virus, comprising depositing the AAV particle in a cryoprotective formulation or pharmaceutical composition embodied herein. In certain embodiments, the formulation is utilized for storing or maintaining viability of the AAV particle over periods of time at temperatures from about 20° C. to about −80° C. In certain embodiments, the AAV particle remains stable over multiple freeze/thaw cycles. In certain embodiments, the AAV particle is stable over multiple freeze-thawing cycles. In certain embodiments, the AAV particle is stable over at least 5 multiple freeze-thawing cycles. In certain embodiments, the AAV particle is stable at temperatures from about 20° C. to about −80° C. In certain embodiments, the AAV particle is stable at about 4° C.

### Stability Assays

As provided herein, the formulations or pharmaceutical compositions of the present disclosure are able to prevent aggregation and/or enhance the stability of the AAV particles. As used herein, a composition or formulation comprising AAV particles is stable if it shows a reduction in viral particle infectivity of less than 50%, less than 40%, less than 30%, less than 20%, less than 10%, or less than 5% upon exposure to a freeze/thaw cycles. As used herein, a composition or formulation comprising AAV particles is stable if is shows a reduction in viral particle infectivity of less than 50%, less than 40%, less than 30%, less than 20%, less than 10%, or less than 5% when stored at a storage temperature of interest for, e.g., at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, at least 6 months, at least 7 months, at least 8 months, at least 9 months, at least 10 months, at least 11 months, or at least 12 months.

Various techniques are known in the art for measuring the physical properties (e.g., AAV particle size), viral titer, and/or purity of the AAV particles. Exemplary assays are provided below, and in some instances, the assay can measure multiple properties. It should be understood, however, that the application is not limited to the assays described below. As provided herein, the formulations and pharmaceutical compositions are able to prevent aggregation and/or enhance the stability of the AAV particle. Various techniques are known in the art for measuring the physical properties (e.g., AAV particle size), viral titer, and/or purity of the AAV particle. Exemplary assays are provided below, and in some instances, the assay can measure multiple properties. It should be understood, however, that the application is not limited to the assays described below.

In certain embodiments, the stability of the AAV particle is measured by a variety of assays, such as dynamic light scattering (DLS), analytical ultracentrifugation (AUC), light microscopy, size exclusion chromatography (SEC), transmission electron microscopy (TEM), field flow fractionation with multi-angle static light scattering (FFF-MALS), infectivity, immunocytochemistry, image analysis and the like.

Various assays are known in the art for evaluating the identity of the AAV particle preparation. In some embodiments, the identity of the AAV particle is derived from an AAV serotype selected from serotypes 1, 2, 3, 4, 5, 6, 7, 8, 9, 10, 11, 12, 13, 14, 15, 16, DJ or DJ/8. In some embodiments, the identity of the AAV particle is AAV2. For example, assays that analyze viral protein expression can be useful in evaluating the identity of the AAV particles. Such assays include, but are not limed to, SDS-PAGE, mass spectrometry, immunoblotting, and ELISA. The proper number, molecular weight, and stoichiometry of the viral proteins can be used to positively identify the vector, as well as the presence of impurities. In addition, the vector genome can be evaluated using PCR or high-throughput NGS (next generation genome sequencing) to ensure positive identity.

AAV particles can also be characterized by their viral titers. Viral titers can include physical titers, as well as functional titers. Physical titer calculates the total number of alive and dead viral particles present and is expressed as the number of viral particles per mL (VP/mL), or for AAV as genome copies per mL (GC/mL). Various methods can be used to determine the physical titer of virus based on quantifying the concentration of viral genomes or viral proteins. Suitable techniques include, but are not limited to, DNA hybridization, real-time PCR—including, but not limited to, quantitative PCR (qPCR), and digital drop PCR (dPCR), optical density (A₂₆₀/₂₈₀), NanoSight, and high-performance liquid chromatography (HPLC).

For example, the Optical Density (A₂₆₀/₂₈₀) assay measures the concentration of viral DNA and protein. It is a physical assay measuring the concentration of viral particles (VP). HPLC is also a rapid method to quantify total viral particles by separation of intact virus particles from other cellular contaminants or virus particle fragments.

Functional titer measures how much virus gets into a target cell and can include assessment of the number of colony forming units following antibiotic selection if the vector contains an antibiotic resistance gene, or, if the vector contains a fluorescent protein, flow cytometry or immunofluorescence analysis of the target cells. Alternatively, if the vector does not express a fluorescent protein, determining the number of integrated proviral DNA copies per cell by qPCR provides a fast and easy method for assessing functional titer.

### EXAMPLES

### Example 1: Designing and Screening Formulations to Improve Manufacturability and Distribution of AAV Gene Therapies

### Materials and Methods

AAV-AQP1 (Aquaporin-1) clarified lysate was by affinity chromatography, split into two pots, and subsequently processed separately by ultrafiltration and diafiltration (UF/DF). The first aliquot was buffer exchanged into Formulation A: 10 mM sodium phosphate, 180 mM NaCl, 1.5% cyclodextrin, 0.02% polysorbate 80 at pH 7.5, whilst the second one was buffer exchanged into Formulation B: 10 mM Tris, 50 mM Mg₂SO₄, 1.5% trehalose, 0.02% polysorbate 80 at pH 7.5. The UF/DF step was performed using an AKTA™ Flux system (Cytiva, Marlborough, MA, USA) with a 115 cm², 100 kDa mPES hollow fiber membrane (Repligen Corporation, Rancho Dominguez, CA, USA). The buffer exchanged and concentrated material was then filtered using a 0.2 μm syringe filter. The volume of 5 mL of the processed material was used to generate different buffer combinations that belonged to either Formulation A (Final buffer compositions were created based on the equations generated from the known buffer components). The targeted ionic strength and osmolality as well as precise buffer components can be found in Table 1 and Table 2.

Formulation A (Table 1) or Formulation B (Table 2) made up the final volume of 20 mL. Stock solutions were prepared to accommodate a wide variety of buffer compositions. The target product concentration was 1×10¹² VG/mL. The samples were aliquoted and held in different storage conditions including room temperature at +20° C., fridge at +4° C., and in freezers at −20° C. and −80° C. over time (Table 3).

### Buffer Design

Buffer ionic strength (I) was calculated as a function of the ions present in solution, i.e., the sum of molar concentration (c) of each ion multiplied by the valence (z) squared, and divided by 2 (Equation 1).

$$I = \frac{1}{2} {{\sum }^{n}}_{i = 1} {c}_{i} {z}_{i}^{2} \qquad Equation 1$$

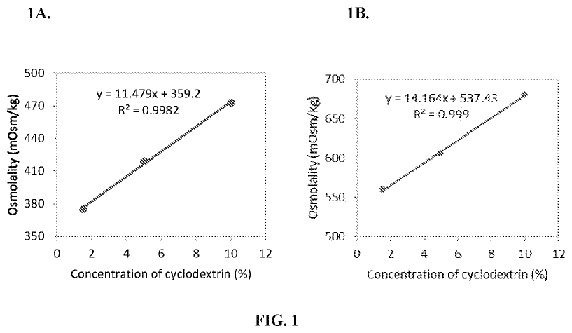

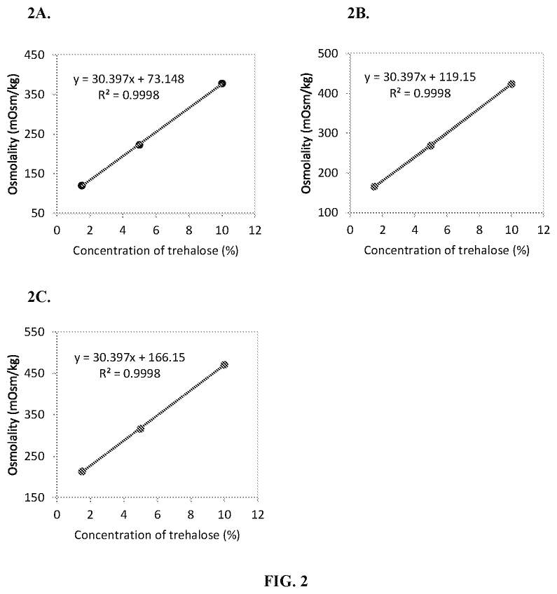

Buffers containing differences in their osmolality were designed based on the ionic strength and saccharide content. Buffers containing a set ionic strength and varying concentration of cyclodextrin or trehalose were created in order to assess the changes to osmolality (FIGS. 1A-1B and FIGS. 2A-2C). The use of three saccharide concentrations allowed to fit a linear line through and generate an equation that could be used to predict the amount of saccharide needed to reach the desired osmolality.

Final buffer compositions were created based on the equations generated from the known buffer components. The targeted ionic strength and osmolality as well as precise buffer components can be found in Table 1 and Table 2.

TABLE 1
Final buffer compositions that belong to Formulation A.

| | Sodium | | Cyclo- | Polysor- | Ionic | |
|---|---|---|---|---|---|---|
| Formula- | phosphate | NaCl | dextrin | bate 80 | strength | Osmolality |
| tion A | (mM) | (mM) | (%) | (%) | (mM) | (mOsm/kg) |
| 1 | 10 | 180 | 0.375 | 0.02 | 200 | 350 |
| 2 | 10 | 180 | 21.0 | 0.02 | 200 | 600 |
| 3 | 10 | 280 | 0.375 | 0.02 | 300 | 350 |
| 4 | 10 | 280 | 11.5 | 0.02 | 300 | 600 |

TABLE 2
Final buffer compositions that belong to Formulation B.

| | | | Treha- | Polysor- | Ionic | |
|---|---|---|---|---|---|---|
| Formula- | Tris | Mg₂SO₄ | lose | bate 80 | strength | Osmolality |
| tion B | (mM) | (mM) | (%) | (%) | (mM) | (mOsm/kg) |
| 1 | 10 | 50 | 4.2 | 0.02 | 200 | 200 |
| 2 | 10 | 50 | 9.1 | 0.02 | 200 | 350 |
| 3 | 10 | 50 | 14.0 | 0.02 | 200 | 500 |
| 4 | 10 | 87.5 | 2.7 | 0.02 | 350 | 200 |
| 5 | 10 | 87.5 | 7.6 | 0.02 | 350 | 350 |
| 6 | 10 | 87.5 | 12.5 | 0.02 | 350 | 500 |
| 7 | 10 | 125 | 1.1 | 0.02 | 500 | 200 |
| 8 | 10 | 125 | 6.0 | 0.02 | 500 | 350 |
| 9 | 10 | 125 | 11.0 | 0.02 | 500 | 500 |

### Hold Study Plan

Samples were held at different temperatures over a set period of time (Table 3) and thawed for 1 hour at room temperature prior to analysis. All analyses were completed on separate days, and thus a new vial was thawed each time. Once the hold time for samples in +20° C. and +4° C. was completed, the samples were placed into −80° C. storage ready for analysis. A separate set of samples was stored in −20° C. and −80° C. and defrosted for 1 hour at room temperature to generate different freeze/thaw cycles.

TABLE 3
First hold study plan for all buffers in Formulations A and B.

| Sample | Temperature | Week | | Freeze/thaw cycle |
|---|---|---|---|---|
| 1 | +20° C. | 24 | hours | 1 |
| 2 | | 48 | hours | |
| 3 | | 1 | week | |
| 4 | +4° C. | 0 | | 1 |
| 5 | | 1 | | |
| 6 | | 2 | | |
| 7 | | 3 | | |
| 8 | | 4 | | |
| 9 | −20° C. | 0 | | 1 |
| 10 | | | | 3 |
| 11 | | | | 6 |
| 12 | | | | 10 |
| 13 | −80° C. | 0 | | 1 |
| 14 | | | | 3 |
| 15 | | | | 6 |
| 16 | | | | 10 |

### Analytical Assays

The vector genome concentration was determined by qPCR.

The viral particle concentration was determined using the Gyrolab® AAVX Titer Kit (Gyros Protein Technologies).

Monomer area and HMW species were determined by HPLC-SEC (Agilent Technologies) using Xbridge BEH450 SEC 3.5 m 7.8×300 mm (Waters™) column.

Infectivity assay was performed by transducing COS-7 cells with the AQP1 product, followed by lysis, single-strand DNA degradation and end point qPCR on a validated AQP1 VG titer assay.

All statistical analysis and model development was performed using JMP® (SAS Institute).

### Results

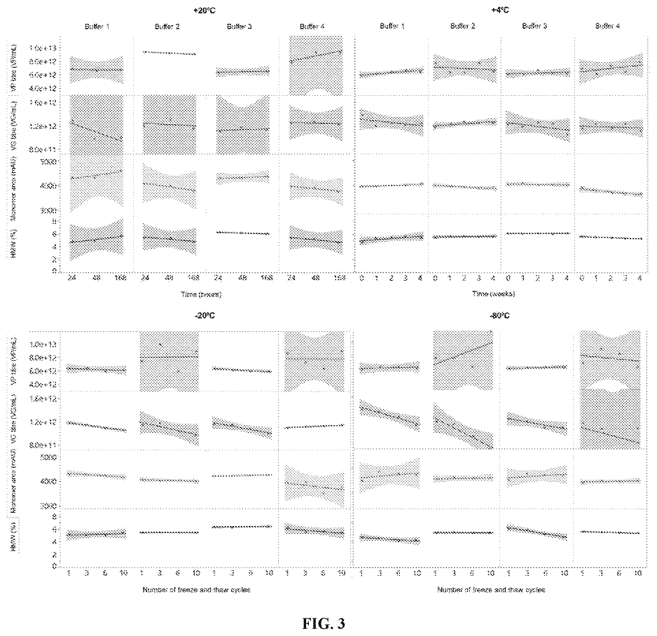

Stability—Formulation A: AAV2 stability was evaluated by measuring VP titer, VG titer, monomer area, presence of high molecular weight species (HMW) before and after the exposure to different storage conditions. Storage in a fridge for up to 4 weeks rather than at room temperature for up to a week (168 hours) was generally found to have less of an impact on the change in the studied parameters (FIG. 3). The effect of product storage at both freezers −20° C. and −80° C. on the stability of AAV2 was found to be comparable. Buffer 3 provided the least amount of variability with a maximum change in VP titer of 7%, VG titer of 15%, monomer area of 5%, and HMW of 22% across all the conditions studied (FIG. 3). The change in HMW was the highest at −80° C. storage, whilst the rest of the conditions resulted in below than 5% difference when using buffer 3.

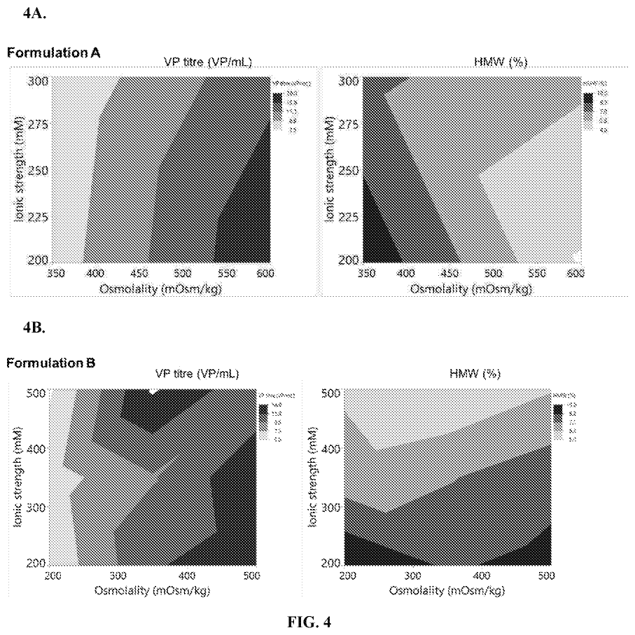

Data summary using relative standard deviation (RSD): In order to summarize the data and ensure that the selected formulation provided the best product stability in terms of VP titer and HMW content across all storage conditions, RSD was utilized. An increase in osmolality led to an increase in VP titer variation for both formulations, whilst an increase in ionic strength had no effect (FIGS. 4A-4B). Specifically, osmolality below 400 mOsm/kg and 250 mOsm/kg was found to provide below 7% variation in VP titer for Formulation A and Formulation B, respectively. In contrast, ionic strength was the key determinant of changes to HMW species whereby an increase in ionic strength led to a decrease in the HMW variability for Formulation B (FIG. 4B). Surprisingly, the same trend was not seen for the Formulation A, in which RSD for HMW decreased with the increase in osmolality with ionic strength showing no significant effects. Overall, maximum percent RSD was found to be 20% for VP titer and 10% for HMW species for both formulations.

### Example 2: Buffer Formulations

### Methods

AAV-AQP1 clarified lysate that was processed through the affinity chromatography step was split into two pots and processed separately during ultrafiltration and diafiltration (UF/DF) steps. The first aliquot was buffer exchanged into Formulation A—Buffer 3: 10 mM sodium phosphate, 280 mM NaCl, 0.4% cyclodextrin, 0.02% polysorbate 80 at pH 7.5, whilst the second one was buffer exchanged into Formulation B—Buffer 7: 10 mM Tris, 125 mM Mg₂SO₄, 1.1% trehalose, 0.02% polysorbate 80 at pH 7.5. The UF/DF step was performed using an Ambr® Crossflow system with a 10 cm², 100 kDa Ambr® CF PESU filter (both Sartorius Stedim UK Limited, Surrey, UK). The buffer exchanged and concentrated material was then filtered using a 0.2 μm syringe filter. The targeted product concentration was 1×10¹¹ VG/mL. The samples were aliquoted and held in different storage conditions (Table 4).

### Hold Study Plan

Hold study plan was executed in the same manner as described in Example 1 with the extended hold time at +20° C. temperature (Table 4).

TABLE 4
Second hold study plan for Formulation
A-Buffer 3 and Formulation B-Buffer 7.

| Sample | Temperature | Week | | Freeze/thaw cycle |
|---|---|---|---|---|
| 1 | +20° C. | 72 | hours | 1 |
| 2 | | 1 | week | |
| 3 | | 2 | weeks | |
| 4 | +4° C. | 0 | | 1 |
| 5 | | 1 | | |
| 6 | | 2 | | |
| 7 | | 3 | | |
| 8 | | 4 | | |
| 9 | −20° C. | 0 | | 1 |
| 10 | | | | 3 |
| 11 | | | | 6 |
| 12 | | | | 10 |
| 13 | −80° C. | 0 | | 1 |
| 14 | | | | 3 |
| 15 | | | | 6 |
| 16 | | | | 10 |

### Results

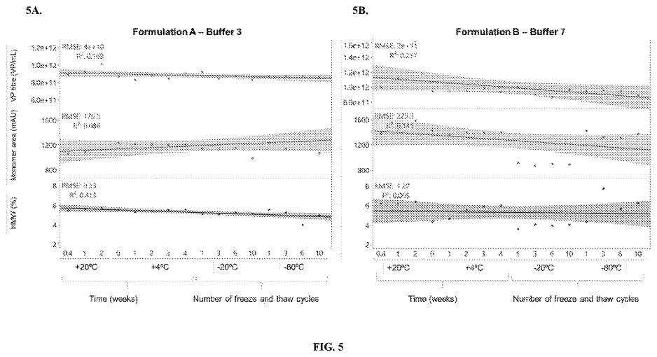

Stability: Based on earlier results (FIGS. 4A-4B), buffer 3 from Formulation A and buffer 7 from Formulation B were selected for the next study that used a 10-fold lower product concentration. AAV2 stability was evaluated by measuring VP titer, monomer area, presence of high molecular weight species before and after the exposure to different storage conditions. Formulation A—buffer 3 had generally a lower variation in the data measured than Formulation B—buffer 7 (FIGS. 5A-5B).

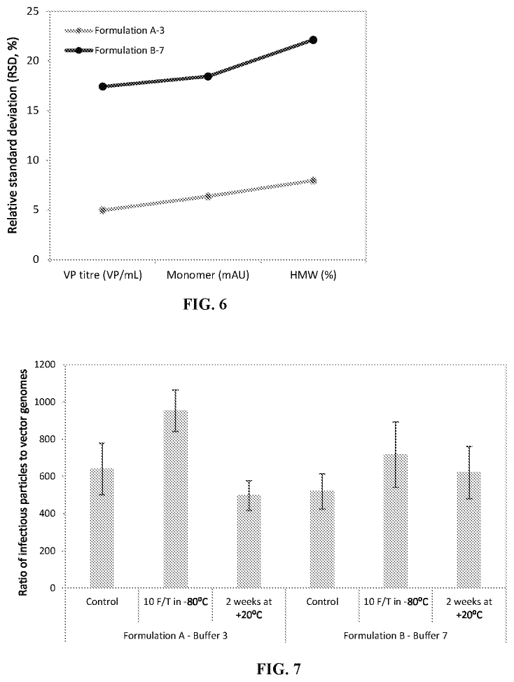

Data summary using relative standard deviation (RSD): Relative standard deviation was calculated for the VP titer, monomer area and HMW content across all storage conditions. Formulation B—buffer 7 had a 4-fold higher RSD than Formulation A—buffer 3 in all assays tested (FIG. 6). Note that in FIG. 6, monomer area result for the Formulation A—Buffer 3 for 6^{th} freeze/thaw cycle at −80° C. of 1802 mAU was deemed to be an outlier as it did not follow any trends and was above the control value and was thus excluded from the RSD calculations. The overall maximum RSD value was below 25% for the Formulation B and below 10% for the Formulation A.

Infectivity: AAV2 infectivity was evaluated for the two formulations stored at room temperature for 2 weeks at +20° C. and in the freezer at −80° C. followed by 10 freeze-thaw (F/T) cycles. The ratio of infectious VP versus total vector genomes (VG) was comparable across the formulation buffers and storage conditions studied (FIG. 7).

Sodium phosphate and sodium chloride-based formulation buffers generally provided a more stable environment for the AAV. Buffer ionic strength of 300-500 mM and osmolality below 400 mOsm/kg water were found to be the most beneficial for AAV2 stability providing the least amount of variation to VP titer, VG titer, monomer area and HMW. The use of relative standard deviation helped to deconvolute complex data sets. Differences in virus concentration could lead to varying stability.

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

What is claimed:

1. A cryoprotective formulation for storing, maintaining infectivity, and/or maintaining viability of adeno-associated virus (AAV) particles, the formulation comprising a saccharide, a salt, a buffer, a surfactant, or combinations thereof.

2. A pharmaceutical composition for storing, maintaining infectivity, and/or maintaining viability of AAV particles comprising a saccharide, a salt, a buffer, a surfactant, or combinations thereof.

3. The cryoprotective formulation of claim 1 or the pharmaceutical composition of claim 2, wherein the saccharide comprises one or more saccharides.

4. The cryoprotective formulation of claims 1 or 3 or the pharmaceutical composition of claims 2 or 3, wherein the one or more saccharides comprise trehalose, cyclodextrin, sucrose, or combinations thereof.

5. The cryoprotective formulation or the pharmaceutical composition of claim 3, wherein the saccharide is cyclodextrin.

6. The cryoprotective formulation or the pharmaceutical composition of claim 5, wherein the cyclodextrin is at a concentration of about 0.1% weight/volume (w/v) up to about 20% (w/v).

7. The cryoprotective formulation or the pharmaceutical composition of claim 6, wherein the cyclodextrin is at a concentration of about 0.1% (w/v) up to about 1% (w/v).

8. The cryoprotective formulation or the pharmaceutical composition of claim 7, wherein the cyclodextrin is at a concentration of about 0.4% (w/v).

9. The cryoprotective formulation or the pharmaceutical composition of claim 3, wherein the saccharide is trehalose.

10. The cryoprotective formulation or the pharmaceutical composition of claim 9, wherein the trehalose is at a concentration of about 0.5% (w/v) to about 20% (w/v).

11. The cryoprotective formulation or the pharmaceutical composition of claim 10, wherein the trehalose is at a concentration of about 0.5% (w/v) to about 5% (w/v).

12. The cryoprotective formulation or the pharmaceutical composition of claim 11, wherein the trehalose is at a concentration of about 1.1% (w/v).

13. The cryoprotective formulation of any one of claims 1, or 3-12 or the pharmaceutical composition of claims 2-12, wherein the salt comprises a sodium salt, a magnesium salt, a calcium salt, a potassium salt, a phosphate salt; a sulfate salt, triethylamine, guanidine, N-substituted guanidine salts, acetamidine, N-substituted acetamidine, pyridine, picoline, ethanolamine, triethanolamine, dicyclohexylamine or N,N′-dibenzylethylenediamine salts, or combinations thereof.

14. The cryoprotective formulation or the pharmaceutical composition of claim 13, wherein the salt is a sodium salt, a magnesium salt, a calcium salt, a potassium salt, a phosphate salt, a sulfate salt or combinations thereof.

15. The cryoprotective formulation or the pharmaceutical composition of claim 14, wherein the salt is a sodium salt.

16. The cryoprotective formulation or the pharmaceutical composition of claim 15, wherein the sodium salt is sodium chloride, sodium phosphate, or both.

17. The cryoprotective formulation or the pharmaceutical composition of claim 16, wherein the sodium chloride is at a concentration of about 100 mM to about 400 mM.

18. The cryoprotective formulation or the pharmaceutical composition of claim 17, wherein the sodium chloride is at a concentration of about 200 mM to about 350 mM.

19. The cryoprotective formulation or the pharmaceutical composition of claim 18, wherein the sodium chloride is at a concentration of about 280 mM.

20. The cryoprotective formulation or the pharmaceutical composition of claim 14, wherein the salt is a magnesium salt.

21. The cryoprotective formulation or the pharmaceutical composition of claim 20, wherein the magnesium salt is magnesium sulfate.

22. The cryoprotective formulation or the pharmaceutical composition of claim 21, wherein the magnesium sulfate is at concentration of about 10 mM to about 250 mM.

23. The cryoprotective formulation or the pharmaceutical composition of claim 22, wherein the magnesium sulfate is at concentration of about 50 mM to about 200 mM.

24. The cryoprotective formulation or the pharmaceutical composition of claim 23, wherein the magnesium sulfate is at concentration of about 125 mM.

25. The cryoprotective formulation of any one of claims 1, or 3-24 or the pharmaceutical composition of claims 2-24, wherein the buffer comprises phosphate buffered saline (PBS), sodium phosphate, citric acid, acetic acid, tromethamine, aspartic acid, glutamic acid, HEPES, Tris, Bicine, acetate, glutamate, lactate, maleate, tartrate, phosphate, citrate, carbonate, glycinate, histidine, glycine, lysine, arginine, succinate, HEPES (4-(2-hydroxyethyl)-1-piperazineethanesulfonic acid), MOPS (3-(N-morpholino) propanesulfonic acid), MES (2-(N-morpholino)ethanesulfonic acid), triethanolamine buffer, or combinations thereof.

26. The cryoprotective formulation or the pharmaceutical composition of claim 25, wherein the buffer comprises sodium phosphate.

27. The cryoprotective formulation or the pharmaceutical composition of claim 26, wherein the sodium phosphate is at a concentration of about 1 mM to about 20 mM.

28. The cryoprotective formulation or the pharmaceutical composition of claim 27, wherein the sodium phosphate is at a concentration of about 10 mM.

29. The cryoprotective formulation or the pharmaceutical composition of claim 25, wherein the buffer comprises Tris.

30. The cryoprotective formulation or the pharmaceutical composition of claim 29, wherein the Tris is at a concentration of about 1 mM to about 20 mM.

31. The cryoprotective formulation or the pharmaceutical composition of claim 30, wherein the Tris is at a concentration of about 10 mM.

32. The cryoprotective formulation of any one of claims 1, or 3-31 or the pharmaceutical composition of claims 2-31, wherein the surfactant is a non-ionic surfactant.

33. The cryoprotective formulation or the pharmaceutical composition of claim 32, wherein the non-ionic surfactant comprises a polysorbate.

34. The cryoprotective formulation or the pharmaceutical composition of claim 33, wherein the polysorbate is polysorbate 80.

35. The cryoprotective formulation or the pharmaceutical composition of any one of claims 32-34, wherein the polysorbate is at a concentration of about 0.01% (w/v) to about 5% (w/v).

36. The cryoprotective formulation or the pharmaceutical composition of claim 35, wherein the polysorbate is at a concentration of 0.001% (w/v) to about 1% (w/v).

37. The cryoprotective formulation or the pharmaceutical composition of claim 36, wherein the polysorbate is at a concentration of 0.02% (w/v).

38. The cryoprotective formulation of any one of claims 1, or 3-37 or the pharmaceutical composition of claims 2-37, wherein the cryoprotective formulation or pharmaceutical composition has an ionic strength of about 100 mM to about 700 mM.

39. The cryoprotective formulation or the pharmaceutical composition of claim 38, wherein the cryoprotective formulation or pharmaceutical composition has an ionic strength of about 200 mM to about 600 mM.

40. The cryoprotective formulation or the pharmaceutical composition of claim 39, wherein the cryoprotective formulation or pharmaceutical composition has an ionic strength of about 300 mM to about 500 mM.

41. The cryoprotective formulation or the pharmaceutical composition of claim 40, wherein the cryoprotective formulation or pharmaceutical composition has an ionic strength of about 300 mM.

42. The cryoprotective formulation or the pharmaceutical composition of claim 40, wherein the cryoprotective formulation or pharmaceutical composition has an ionic strength of about 500 mM.

43. The cryoprotective formulation of any one of claims 1, or 3-42 or the pharmaceutical composition of claims 2-42, wherein the cryoprotective formulation or pharmaceutical composition has an osmolality from about 100 mOsm/kg to about 800 mOsm/kg.

44. The cryoprotective formulation or the pharmaceutical composition of claim 43, wherein the cryoprotective formulation or pharmaceutical composition has an osmolality from about 200 mOsm/kg to about 600 mOsm/kg.

45. The cryoprotective formulation or the pharmaceutical composition of claim 43, wherein the cryoprotective formulation or pharmaceutical composition has an osmolality of less than about 400 mOsm/kg.

46. The cryoprotective formulation or the pharmaceutical composition of claim 43, wherein the cryoprotective formulation or pharmaceutical composition has an osmolality of about 200 mOsm/kg.

47. The cryoprotective formulation or the pharmaceutical composition of claim 43, wherein the cryoprotective formulation or pharmaceutical composition has an osmolality of about 350 mOsm/kg.

48. The cryoprotective formulation of any one of claims 1, or 3-47 or the pharmaceutical composition of claims 2-47, wherein the cryoprotective formulation or pharmaceutical composition has a pH of about 7.0 to about 8.0.

49. The cryoprotective formulation or the pharmaceutical composition of claim 48, wherein the cryoprotective formulation or pharmaceutical composition has a pH of about 7.5.

50. The cryoprotective formulation of any one of claims 1 or 38-49, or the pharmaceutical composition of any one of claims 2 or 38-49, wherein the cryoprotective formulation or pharmaceutical composition comprises (1) cyclodextrin from about 0.1% (w/v) up to about 20% (w/v), (2) sodium chloride from about 100 mM to about 400 mM, (3) sodium phosphate from about 1 mM to about 20 mM, and (4) polysorbate from 0.001% (w/v) to about 1% (w/v).

51. The cryoprotective formulation or the pharmaceutical composition of claim 50, wherein the cryoprotective formulation or pharmaceutical composition comprises (1) cyclodextrin at about 0.4% (w/v), (2) sodium chloride at about 280 mM, (3) sodium phosphate at about 10 mM, and (4) polysorbate 80 at about 0.02% (w/v).

52. The cryoprotective formulation of any one of claims 1 or 38-49, or the pharmaceutical composition of any one of claims 2 or 38-49, wherein the cryoprotective formulation or pharmaceutical composition comprises (1) trehalose from about 1% (w/v) to about 20% (w/v), (2) magnesium sulfate from about 10 mM to about 250 mM, (3) Tris from about 1 mM to about 20 mM, and (4) polysorbate from 0.001% (w/v) to about 1% (w/v).

53. The cryoprotective formulation or the pharmaceutical composition of claim 52, wherein the cryoprotective formulation or pharmaceutical composition comprises (1) trehalose at about 1.1% (w/v), (2) magnesium sulfate at about 125 mM, (3) Tris at about 10 mM, and (4) polysorbate 80 at about 0.02% (w/v).

54. The cryoprotective formulation of any one of claims 1, or 3-53 or the pharmaceutical composition of claims 2-53, the cryoprotective formulation or the pharmaceutical composition further comprises one or more of a pharmaceutical agent, medium, protein, or combinations thereof.

55. The cryoprotective formulation of any one of claims 1, or 3-54 or the pharmaceutical composition of claims 2-54, wherein the AAV particles are derived from an AAV serotype selected from serotypes 1, 2, 3, 4, 5, 6, 7, 8, 9, 10, 11, 12, 13, 14, 15, 16, DJ or DJ/8.

56. The cryoprotective formulation of any one of claims 1, or 3-54 or the pharmaceutical composition of claims 2-54, wherein the AAV particles comprise a genome derived from AAV serotype 2.

57. The cryoprotective formulation of any one of claims 1, 3-54, or 56 or the pharmaceutical composition of claims 2-54 or 56, wherein the AAV particles are comprise a capsid derived from AAV serotype 2.

58. A method of storing or maintaining AAV particle viability and/or AAV particle infectivity at various temperatures, the method comprising depositing the AAV particle into the cryoprotective formulation of any one of claims 1, or 3-54 or the pharmaceutical composition of claims 2-54.

59. The method of claim 58, wherein the AAV particle is derived from an AAV serotype selected from serotypes 1, 2, 3, 4, 5, 6, 7, 8, 9, 10, 11, 12, 13, 14, 15, 16, DJ or DJ/8.

60. The method of claim 58, wherein the AAV particle is comprises a genome derived from AAV serotype 2.

61. The method of claims 58 or 60, wherein the AAV particle comprises a capsid derived from AAV serotype 2.

62. The method of any one of claims 58-61, wherein the AAV particle is stable over multiple freeze-thawing cycles.

63. The method of any one of claims 58-61, wherein the AAV particle is stable over at least 5 freeze-thawing cycles.

64. The method of any one of claims 58-63, wherein the AAV particle is stable at temperatures from about +20° C. to about −80° C.

65. The method of any one of claims 58-63, wherein the AAV particle is stable at a temperature of about +4° C.

66. The method of claim 65, wherein the AAV particle is stable for at least six months at +4° C.

67. The method of claim 65, wherein the AAV particle is stable for at least one year at +4° C.
